(19) United States
(12) Patent Application Publication
(10) Pub. No.: US 2026/0130753 A1
(43) Pub. Date: May 14, 2026

(54) BONE PLATING SYSTEM FOR A LATERAL ULNA BONE

(71) Applicant: Endeavor Orthopaedics, LLC, Tulsa, OK (US)

(72) Inventors: Ryan Christopher Stafford, Fort Wayne, IN (US); Dana James Funk, Tulsa, OK (US); Gregory B. Parranto, JR., Naperville, IL (US); Matthew P. Gardner, Springfield, IL (US)

(21) Appl. No.: 19/385,071

(22) Filed: Nov. 10, 2025

Related U.S. Application Data
(60) Provisional application No. 63/718,008, filed on Nov. 8, 2024.
(60) Provisional application No. 63/883,281, filed on Sep. 17, 2025.

(51) Int. Cl. A61F 2/08; A61B 17/04; A61B 17/80

(52) U.S. Cl. CPC A61F 2/0811; A61B 17/0401; A61B 17/8033; A61B 17/8061; A61B 2017/0414; A61B 2017/0459; A61B 2017/0464; A61F 2002/0817; A61F 2002/0852; A61F 2002/0888

(57) ABSTRACT

The invention provides a lateral ulna bone fixation plate provides rigid fixation of olecranon and proximal-ulna fractures while enabling knotless soft-tissue reattachment within a single integrated implant. The plate body is anatomically contoured to conform to the lateral surface of the proximal ulna and wrap partially around the olecranon to reduce hardware prominence and postoperative soft-tissue irritation. The plate includes a plurality of locking or non-locking screw holes for fracture fixation and one or more suture attachment features defining a slip-knot pathway formed by holes, tunnels, or slots. The slip-knot pathway permits a suture to move freely in one direction for tensioning and resists movement in an opposite direction, establishing a self-locking, knotless construct for reattachment of the triceps tendon or other soft tissue. The lateral positioning and contoured geometry provide improved biomechanical resistance to bending and torsional forces while maintaining a low-profile, anatomically optimized configuration.

### CROSS-REFERENCE TO RELATED APPLICATIONS

This application claims priority to U.S. Provisional Application No. 63/718,008, filed Nov. 8, 2024, and U.S. Provisional Application No. 63/883,281, filed Sep. 17, 2025, which are herein incorporated by reference in their entirety.

### TECHNICAL FIELD

The present invention relates generally to orthopedic fixation systems and, more specifically, to bone plating systems configured to stabilize fractures at anatomical locations influenced by muscular or tendinous forces. The invention encompasses integrated suture and plate constructs that provide knotless fixation and controlled tensioning for soft-tissue reattachment, with particular application to avulsion-type fractures such as those involving the olecranon, proximal ulna, patella, and other regions subjected to extensor mechanism loading.

### BACKGROUND OF THE INVENTION

Fractures occurring at anatomical sites that experience tensile loading from tendons or ligaments, such as the olecranon, patella, greater tuberosity of the humerus, and greater trochanter of the femur, pose significant challenges in achieving stable fixation. Traditional plate-and-screw constructs often fail to adequately neutralize deforming forces generated by attached muscles or tendons. For example, in olecranon fractures, the triceps tendon exerts tension that can displace fracture fragments even after internal fixation, increasing the likelihood of fixation failure or loss of reduction.

Existing fixation methods often require secondary devices such as tension-band wires or cables to counteract these forces. These techniques increase surgical complexity and can lead to complications including hardware irritation, knot prominence, and the need for hardware removal. Furthermore, knot-tying introduces inconsistency in tension control and mechanical performance, as it depends heavily on surgeon technique and intraoperative conditions.

Accordingly, there remains a need for an integrated bone plating system that provides (1) rigid internal fixation; (2) controlled and reproducible soft-tissue tensioning; and (3) a low-profile, knotless construct that minimizes soft-tissue irritation and streamlines the surgical procedure.

### SUMMARY OF THE INVENTION

The present invention provides an integrated orthopedic fixation system that enhances the precision, reliability, and biomechanical performance of soft-tissue and bone fixation constructs. The system combines novel suture and plate technologies designed to address the limitations of traditional knot-based fixation methods and posteriorly positioned bone plates. In particular, the invention comprises three complementary innovations: a loop suture for controlled tensioning, a bone plate with an integrated slip-knot pathway, and a lateral ulna plate configured for improved anatomical conformity and soft-tissue compatibility.

In one aspect, the invention provides a loop suture designed to enhance surgeon control and precision during soft-tissue fixation procedures involving tendons, ligaments, or muscles. The loop suture includes one or more pre-formed, integrally constructed loops along its length, each functioning as a selectable tensioning point. By engaging one or more of these loops, the surgeon can incrementally “dial in” a precise amount of tension across the repaired tissue. This configuration provides reproducible and quantifiable control of tissue compression, minimizing the risk of overtightening, tissue strangulation, or slack. The loop suture may include a pre-attached surgical needle at one end to facilitate passage through soft tissue, while the opposite end may attach to a bone plate or implant through either a fixed or adjustable interface. The evenly spaced, integral loops allow for multiple levels of engagement and fine-tuned tension adjustment using a single suture strand, simplifying the surgical procedure and reducing variability in fixation performance.

In another aspect, the invention provides a bone plate with an integrated slip-knot pathway, enabling a suture or flexible member to be routed through the plate in a manner that provides directional locking and self-fixation, forming a secure, knotless construct. The slip-knot pathway is defined by one or more entry and exit holes connected by angled or curved tunnels or slots that guide the suture through the plate body. The internal geometry of the pathway controls frictional engagement between the suture and plate surfaces, allowing the suture to move freely in one direction during tensioning but resist movement in the reverse direction. In one embodiment, the suture crosses itself within or against the plate, further enhancing locking performance by creating a self-binding effect under load. This integrated slip-knot feature eliminates the need for external knot tying, reduces operative time, minimizes knot-related soft-tissue irritation, and allows intraoperative re-tensioning for optimal biomechanical performance.

In a further aspect, the invention provides a lateral ulna plate specifically designed for fixation of olecranon and proximal ulna fractures. Unlike conventional posterior plates that can cause postoperative discomfort and hardware irritation due to limited soft-tissue coverage, the lateral ulna plate is anatomically contoured for placement along the lateral surface of the ulna, where soft-tissue coverage is more substantial. The plate includes a plurality of locking and non-locking screw holes for rigid fixation of fracture fragments and one or more K-wire holes for provisional alignment. A key feature of the lateral ulna plate is the incorporation of the aforementioned slip-knot suture pathway, enabling knotless reattachment of the triceps tendon or other soft tissues under controlled, reproducible tension. The lateral orientation of the plate also aligns the fixation construct with the principal biomechanical loading vectors generated by the triceps during elbow extension, thereby enhancing resistance to bending and torsional forces while maintaining a low-profile implant design.

Together, these innovations provide a comprehensive fixation system that allows surgeons to achieve stable bone fixation, controlled soft-tissue tensioning, and knotless construct integrity in a single integrated platform. The combination of the loop suture and the slip-knot plate mechanism enables reproducible, precise, and efficient intraoperative tension control, while the lateral ulna plate geometry improves implant comfort, reduces hardware prominence, and enhances postoperative outcomes. The invention thus represents a significant advancement in orthopedic fixation technology by integrating controlled tensioning mechanics, knotless fixation, and anatomically optimized plate design into a unified system.

According to an exemplary embodiment, a lateral ulna bone fixation plate may comprise: a plate body anatomically contoured to conform to a lateral surface of a proximal ulna and configured to wrap partially around an olecranon; a plurality of screw holes extending through the plate body and configured to receive locking or non-locking screws for fixation of olecranon and proximal-ulna fracture fragments; at least one suture attachment feature comprising one or more holes, tunnels, or slots configured to receive a suture or flexible member and form a slip-knot pathway that allows the suture to move in a first direction for tensioning and resist movement in an opposite direction, thereby maintaining a knotless self-locking configuration for soft-tissue fixation. The plate body may be positioned on the lateral aspect of the ulna to reduce soft-tissue irritation and provide rigid fixation while minimizing implant prominence relative to posteriorly positioned plates.

Another aspect may include the plate body that includes a proximal portion configured to hook over the olecranon to provide proximal fixation and tendon capture. The suture attachment feature may be configured to enable a pre-attached suture to engage and secure a triceps muscle or tendon to the plate to facilitate healing. The slip-knot pathway may comprise a series of strategically oriented holes, tunnels, and slots configured to guide the suture through the plate and promote unidirectional movement for tensioning. A geometry of the holes, tunnels, and slots may be oriented to increase frictional engagement between the suture and one or more plate walls, thereby enhancing self-locking behavior. The suture may cross itself within or along the plate body to produce a self-binding interaction that resists loosening once tension is applied. The plate body may include two or more suture attachment locations disposed along opposite ends of the plate. The plate body may further include one or more K-wire holes configured for provisional fixation and intraoperative reduction of fracture fragments. The plurality of screw holes may comprise variable-angle locking holes configured to receive screws at a range of insertion angles. The plate body may be formed of a biocompatible metal or composite material selected from the group consisting of titanium, stainless steel, cobalt-chromium alloy, and reinforced polymer composites. The plate body may be low-profile and includes radiused or smoothed edges to minimize interference with surrounding soft tissue. The plate body may be configured to provide biomechanical resistance to bending and torsional forces generated by a triceps during elbow extension. The slip-knot pathway may enable adjustable intraoperative tensioning of the suture prior to final fixation. A plate geometry, curvature, and hole pattern may be modularly configurable to conform to different bone surfaces while maintaining knotless tensioning capability. The plate body may further include identifiable dorsal and volar surfaces, the volar surface comprising a bone-contacting surface adapted to interface with the ulna. The slip-knot suture pathway and plate configuration together may enable combined fracture fixation and soft-tissue reattachment within a single integrated implant.

According to another exemplary embodiment, a method of treating an olecranon or proximal-ulna fracture may comprise: positioning the lateral ulna bone fixation plate according to claim 1 on a lateral surface of the proximal ulna such that the plate wraps partially around the olecranon; fixing the plate to the bone using a plurality of bone screws; routing a suture through the slip-knot pathway of the plate and through a triceps tendon or muscle; tensioning the suture in a first direction to secure soft tissue against the plate while preventing reverse movement; and locking the suture in place without forming an external knot. The plate may be applied to a lateral olecranon region to reduce posterior hardware prominence and minimize postoperative soft-tissue irritation.

According to another exemplary embodiment, a bone fixation plate configured for placement on a lateral surface of a proximal ulna may comprise: a plate body anatomically contoured to the lateral surface of the proximal ulna; a plurality of locking screw holes arranged for rigid fixation of olecranon and proximal-ulna fracture fragments; at least one K-wire hole for provisional fixation; and an integrated suture pathway configured as a slip-knot feature that permits unidirectional movement of a suture for tensioning and resists reverse movement, enabling knotless reattachment of a triceps tendon to the bone. The suture pathway may be oriented to oppose a tensile vector of a triceps mechanism. The plate may include smooth and radiused edges to reduce soft-tissue irritation. The lateral positioning of the plate may provide reduced prominence relative to posterior fixation plates.

Other features and advantages of the invention will be apparent from the following specification taken in conjunction with the following drawings.

### BRIEF DESCRIPTION OF DRAWINGS

The following Detailed Description will be better understood when considered in conjunction with the accompanying drawings in which like reference numerals refer to the same or similar elements in all of the various views in which that reference number appears.

FIG. 1A depicts a perspective view of an exemplary one-hole bone plate with a screw and screw-driver, according to one or more aspects described herein.

FIG. 1B depicts a perspective view of the bone plate from FIG. 1B, according to one or more aspects described herein.

FIG. 1C depicts a perspective view of another exemplary one-hole bone plate with a screw, according to one or more aspects described herein.

FIG. 2 depicts a perspective view of a two-hole bone plate with a screw, according to one or more aspects described herein.

FIG. 3A depicts a perspective view of a four-hole bone plate, according to one or more aspects described herein.

FIG. 3B depicts a top view of the four-hole bone plate from FIG. 3A, according to one or more aspects described herein.

FIG. 3C depicts another perspective view of the four-hole bone plate from FIG. 3A, according to one or more aspects described herein.

FIGS. 4A-4D depict various views of a suture attachment of an exemplary bone plate, according to one or more aspects described herein.

FIG. 4E depicts a cross-sectional view of the suture attachment from FIGS. 4A-4D, according to one or more aspects described herein.

FIG. 4F depicts another cross-sectional view of the suture attachment from FIGS. 4A-4D, according to one or more aspects described herein.

FIGS. 5A-5B depict various views of a cleat of an exemplary bone plate, according to one or more aspects described herein.

FIG. 6A depicts a perspective view of a lateral ulna bone plate, according to one or more aspects described herein.

FIG. 6B depicts a view of the lateral ulna bone plate from FIG. 6A attached via screws on the lateral ulna bone, according to one or more aspects described herein.

FIG. 7A depicts a perspective view of an exemplary bone plate with a loop suture, according to one or more aspects described herein.

FIG. 7B depicts a perspective view of an exemplary patella plate with a loop suture, according to one or more aspects described herein.

FIG. 7C depicts a top view of another loop suture for use with a bone plating system, according to one or more aspects described herein.

FIG. 8 depicts a perspective view of a bone plate system with a bone plate and sutures, according to one or more aspects described herein.

FIG. 9 depicts a perspective view of another bone plate system with a bone plate and a suture, according to one or more aspects described herein.

FIGS. 10A and 10B depict perspective views of another bone plate system with a bone plate, sutures, and screws attached to the bone plate, according to one or more aspects described herein.

Further, it is to be understood that the drawings may represent the scale of different components of one single embodiment; however, the disclosed embodiments are not limited to that particular scale.

### DETAILED DESCRIPTION

In the following description of various examples of the invention, reference is made to the accompanying drawings, which form a part hereof, and in which are shown by way of illustration various example parts, structures, systems, and steps in which aspects of the invention may be practiced. It is to be understood that other specific arrangements of example parts, structures, systems, and steps may be utilized, and structural and functional modifications may be made without departing from the scope of the present invention. Also, while the terms “top,” “bottom,” “front,” “back,” “side,” “rear,” and the like may be used in this specification to describe various example features and elements of the invention, these terms are used herein as a matter of convenience, e.g., based on the example orientations shown in the figures. Nothing in this specification should be construed as requiring a specific three-dimensional orientation of structures in order to fall within the scope of this invention.

This application and/or claims may use the adjectives, e.g., “first,” “second,” “third,” and the like, to identify certain components and/or features relating to this technology. These adjectives are used merely for convenience, e.g., to assist in maintaining a distinction between components and/or features of a specific structure. Use of these adjectives should not be construed as requiring a specific order or arrangement of the components and/or features being discussed. Also, use of these specific adjectives in the specification for a specific structure does not require that the same adjective be used in the claims to refer to the same part (e.g., a component or feature referred to as the “fourth” in the specification may correspond to any numerical adjective used for that component or feature in the claims).

The present invention provides an integrated orthopedic fixation system that combines a novel looped suture, an anatomically contoured bone plate incorporating a slip-knot pathway, and a specialized lateral ulna plate for fracture fixation. The system is designed to address the biomechanical challenges of fractures influenced by extensor mechanisms and to simplify soft-tissue reattachment by enabling reproducible, knotless tensioning directly through the implant construct.

In general, the invention allows controlled transfer of tensile forces exerted by tendons, ligaments, or muscles onto the bone plate rather than the fracture interface, thereby maintaining reduction and promoting anatomical healing. The system may be used for fixation of fractures such as those of the olecranon, proximal ulna, patella, or other regions where soft-tissue attachment and deforming forces complicate traditional fixation.

The invention provides an integrated orthopedic fixation system that enhances precision, reproducibility, and biomechanical stability in bone and soft-tissue repair. In one aspect, a loop suture includes one or more integrally formed loops spaced along its length, each functioning as a selectable tensioning point that enables a surgeon to incrementally “dial in” soft-tissue tension without manual knot-tying. In another aspect, a bone plate with a slip-knot pathway incorporates strategically oriented holes, tunnels, and slots configured to guide a suture in a unidirectional, self-locking manner, forming a stable, knotless construct. In a further aspect, a lateral ulna plate is anatomically contoured for placement on the lateral surface of the proximal ulna and integrates the slip-knot suture pathway to provide rigid fracture fixation while minimizing hardware prominence and optimizing triceps-tendon tension control. The combined system allows precise, repeatable, and adjustable fixation of bone and soft tissue using a single low-profile implant platform.

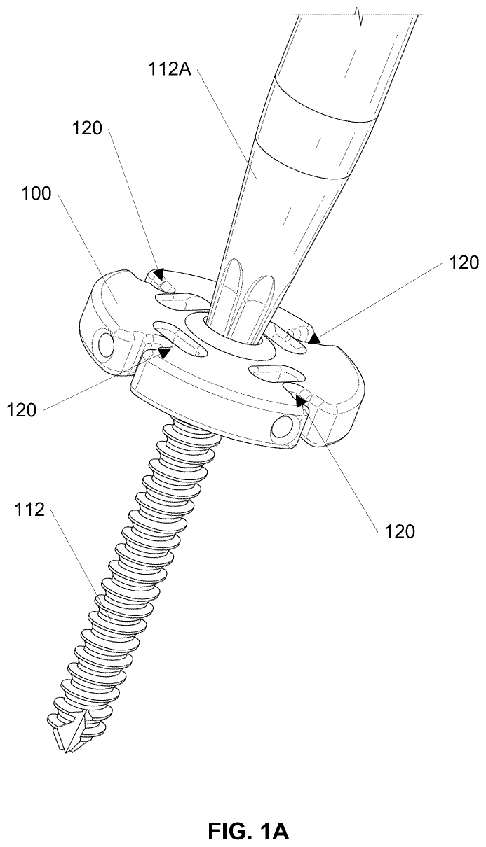

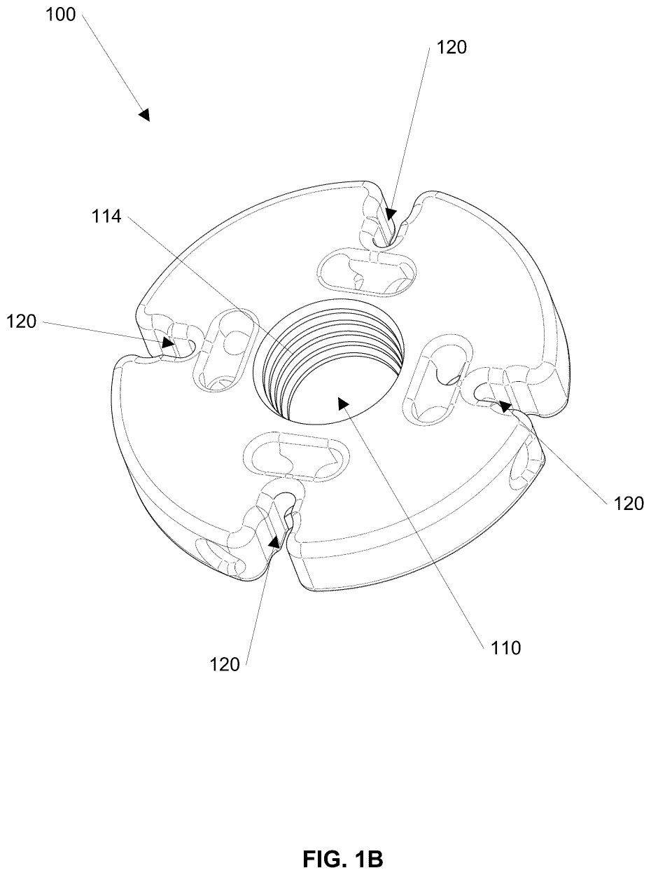

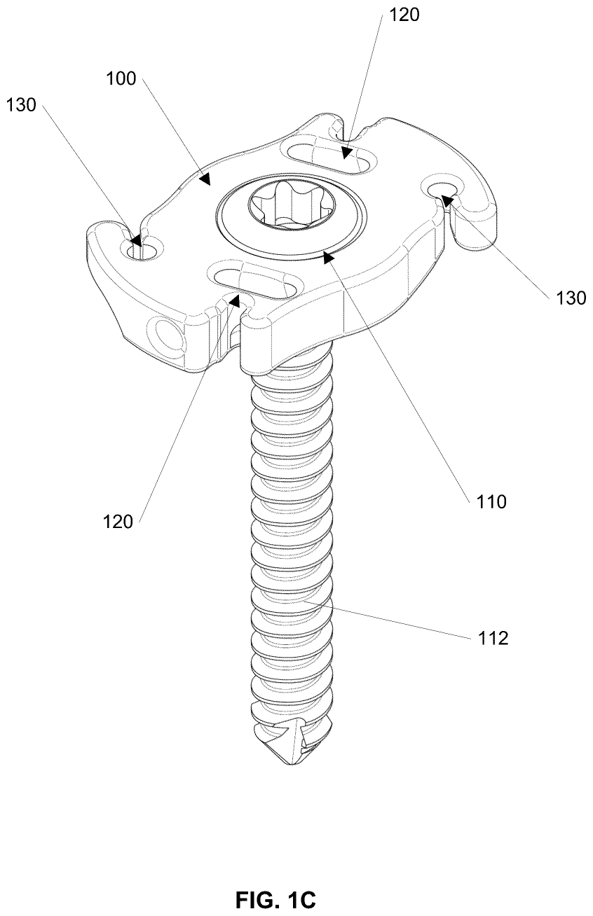

FIGS. 1A-3C depict exemplary bone plates with holes and tunnels that allow sutures to be attached to the bone plate with the holes for screws. The bone plates may be low-profile and capable of contouring to the bone. The bone plates may include at least one set of holes and tunnels that allow for a suture or other flexible device to pass through and attach to the bone plate and configured to create one end that passes through soft tissue and a second end that can be pulled to remove slack. Additionally, the set of tunnels may be configured in such a way that the suture or other flexible device can only be pulled in one direction for the purpose of maintaining tension between the soft tissue and the bone plate. The bone plates may include a second set of holes and tunnels that allow the free end of the suture or other flexible device to securely attach to the bone plate. The combination of the holes and tunnels may allow for the suture or other flexible device to be attached to the bone plate in such a way that will allow for unidirectional movement of the suture or flexible device through the first set of holes and tunnels in the bone plate.

Specifically, FIGS. 1A-1C depict an exemplary embodiment of a bone plate 100 with one locking hole 110. The locking hole 110 may include threads 114 to mate with threads on the locking screw 112. The bone plate 100 may include a plurality of suture attachments 120. In the depicted embodiment of the bone plate 100, the bone plate 100 may include four suture attachments 120. The bone plate 100 with one locking hole 110 may be similar to but not meant to work in place of a suture button or suture anchor. The bone plate 100 with one locking hole 110 may be meant to address avulsion fractures. The bone plate 100 with one locking hole 110 may allow a secure anchor point into bone with a traditional screw 112 followed by the ability secure the tendon with the suture.

FIG. 1A depicts a locking bone plate 100 with one locking hole 110, four suture attachments 120, shown with the locking screw 112 and a screw driver 112A. FIG. 1B depicts the locking bone plate 100 with one locking hole 110, and four suture attachments 120. The suture attachments 120 may be located in the corners of the locking bone plate 100. FIG. 1C depicts another locking bone plate 100A with one locking hole 110, two suture attachment points 120, two cleats 130, and shown with the locking screw 112. The suture attachments 120 and cleats 130 may be located in the corners of the locking bone plate 100A. In the exemplary embodiment shown in FIG. 1C, the suture attachments 120 may be located on opposing corners of the locking bone plate 100A with the cleats 130 located on the other corners of the locking bone plate 100A. Various number of suture attachment points 120 and cleats 130 may be utilized with the locking bone plate 100A. FIGS. 1A-1C do not show any pre-attached sutures.

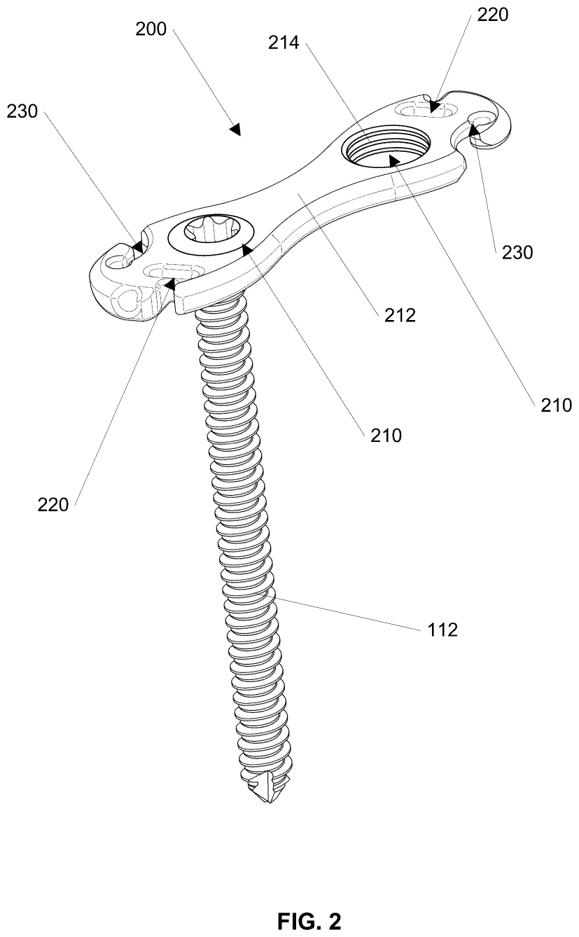

FIG. 2 depicts an exemplary bone plate 200 with two locking holes 210 and a locking screw 112. For the embodiment of FIG. 2, the features of the bone plate 200 are referred to using similar reference numerals under the “2XX” series of reference numerals, rather than “1XX” as used for the bone plate 100, 100A in the embodiments of FIGS. 1A-1C. Accordingly, certain features of the bone plate 200 that were already described above with respect to the bone plate 100, 100A of FIGS. 1A-1C may be described in lesser detail, or may not be described at all. The two-hole bone plate 200 may be essentially used in the same way as the one-hole plate 100, 100A but may allow for bone fracture reduction fixation in areas with enough space to fit a longer plate and two screws.

The bone plate 200 may include two locking holes 210 separated by a plate junction 212. The two locking holes 210 may include threads 214 to mate with threads on the locking screw 112. The bone plate 200 may include two suture attachments 220 and two suture cleats 230. The suture attachments 220 and cleats 230 may be located on the ends of the locking bone plate 200. One suture attachment 220 and one cleat 230 may be located on a first end 202 of the bone plate 200 and a second suture attachment 220 and second cleat 230 may be located on a second end 204 opposite of the first end 202 of the bone plate 200. Various number of suture attachments 220 and cleats 230 may be utilized with the locking bone plate 200.

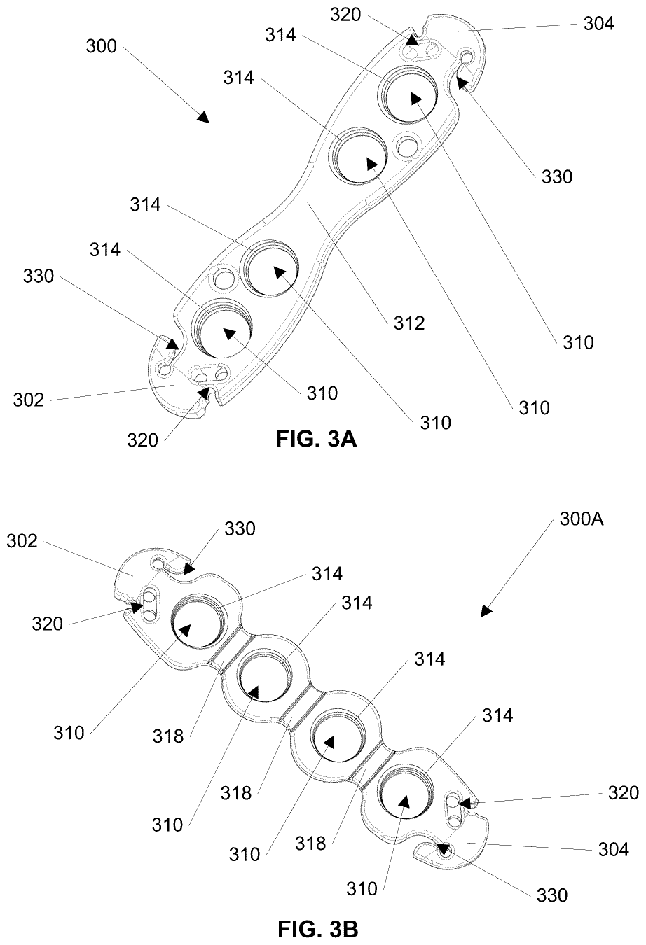

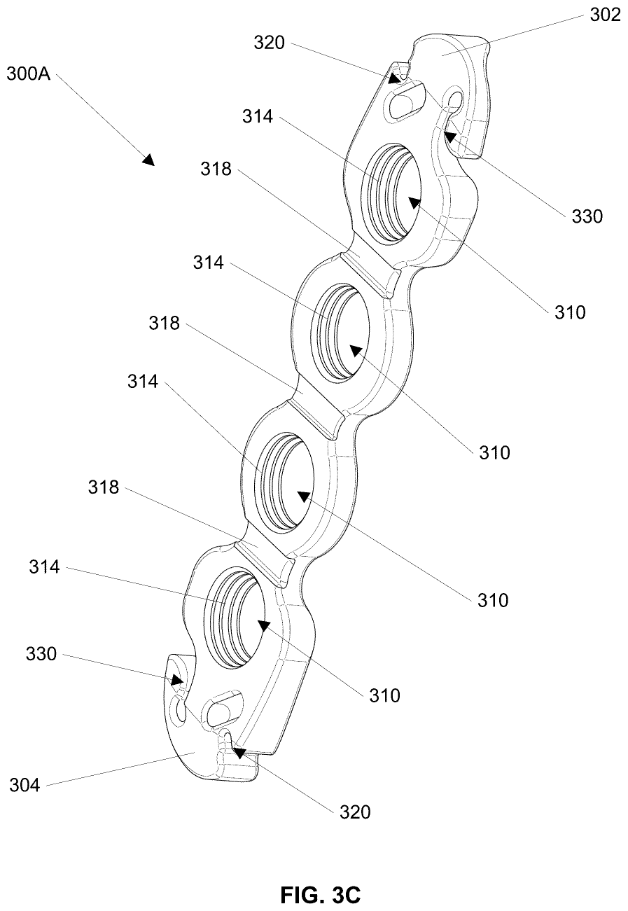

FIGS. 3A-3C depict another exemplary bone plate 300, 300A with four locking holes 310. For the embodiment of FIGS. 3A-3C, the features of the bone plate 300, 300A are referred to using similar reference numerals under the “3XX” series of reference numerals, rather than “1XX” as used for the bone plate 100, 100A in the embodiments of FIGS. 1A-1C and “2XX” as used for the bone plate 200 in the embodiment of FIG. 2. Accordingly, certain features of the bone plate 300, 300A that were already described above with respect to the bone plate 100, 100A of FIGS. 1A-1C and the bone plate 200 of FIG. 2 may be described in lesser detail, or may not be described at all.

The bone plate 300, 300A may include four locking holes 310 for screws 112 (not shown). The bone plate 300, 300A is essentially the same as the one-hole bone plate 100, 100A and two-hole bone plate 200. FIG. 3A depicts a bone plate 300 that is rigid, while FIG. 3B depicts a bone plate 300A that is bendable for irregular shaped boney surfaces and is longer when room is available and more screw fixation is desired.

The bone plate 300 shown in FIG. 3A may include four locking holes 310, with two locking holes 310 on each end of the bone plate 300 separated by a plate junction 312. The four locking holes 310 may include threads 314 to mate with threads on the locking screw 112. The bone plate 300 may include two suture attachments 320 and two suture cleats 330. The suture attachments 320 and cleats 330 may be located on the ends of the locking bone plate 300. One suture attachment 320 and one cleat 330 may be located on a first end 302 of the bone plate 300 and a second suture attachment 320 and second cleat 330 may be located on a second end 304 opposite of the first end 302 of the bone plate 300. Various number of suture attachments 320 and cleats 330 may be utilized with the locking bone plate 300. The first end 302 may include two of the locking holes 310 with the second end 304 include two locking holes 310 separated by the plate junction 312. The bone plate 300 may also include one or more k-wire holes 316, as depicted in FIG. 3A.

The bone plate 300A shown in FIGS. 3B and 3C may include four locking holes 310. Each of the four locking holes 310 may be separated by a bendable junction 318. The bone plate 300A may be bendable and configured to be bent between the locking holes 310 at the bendable junction 318. The four locking holes 310 may include threads 314 to mate with threads on the locking screw 112. The bone plate 300A may include two suture attachments 320 and two suture cleats 330 similar to the bone plate 300, with one suture attachment 320 and one cleat 330 located on a first end 302 of the bone plate 300A and a second suture attachment 320 and second cleat 330 located on a second end 304 opposite of the first end 302 of the bone plate 300A. Various number of suture attachments 320 and cleats 330 may be utilized with the locking bone plate 300A.

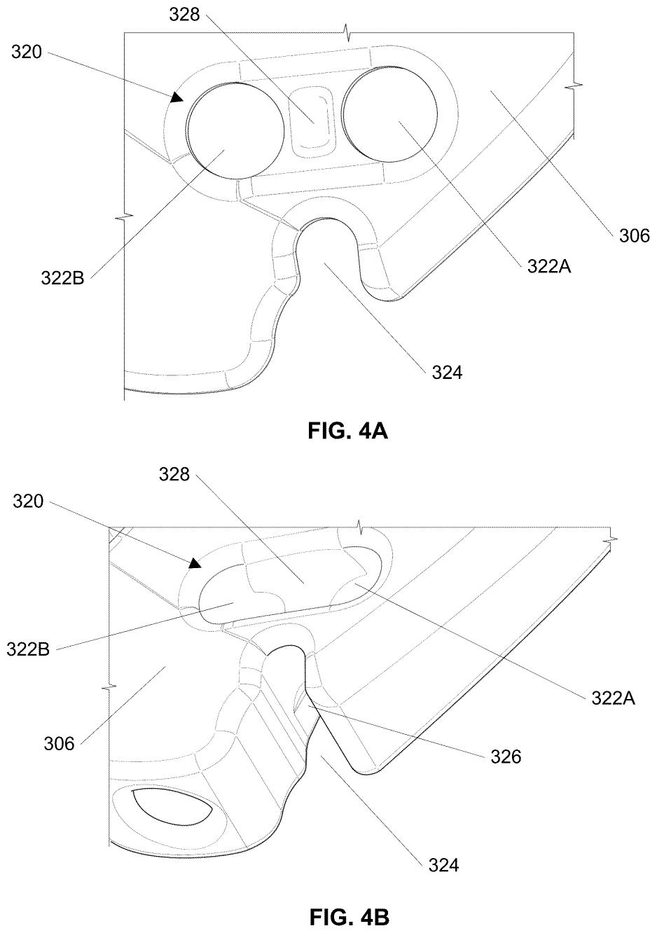

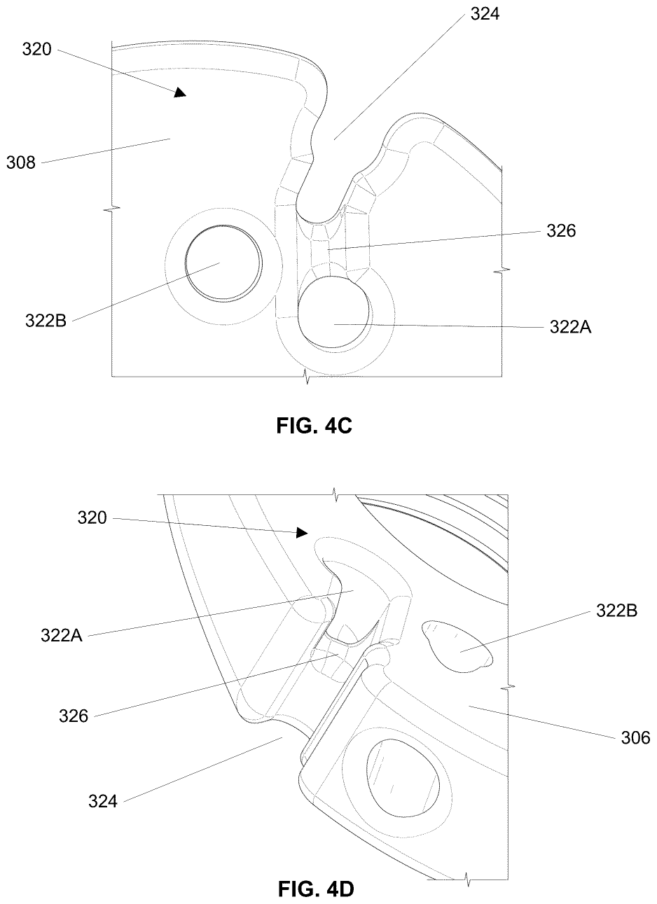

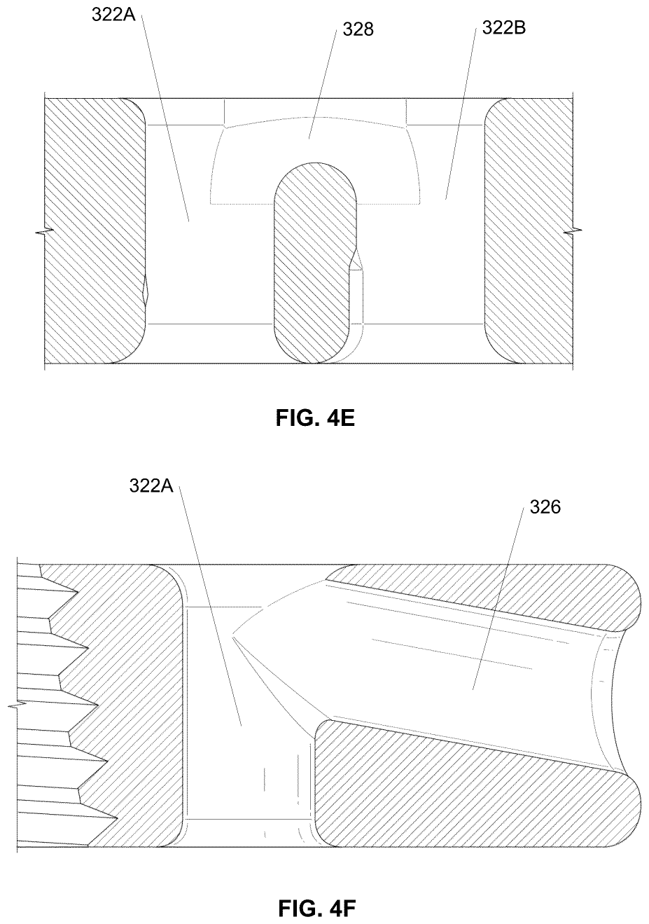

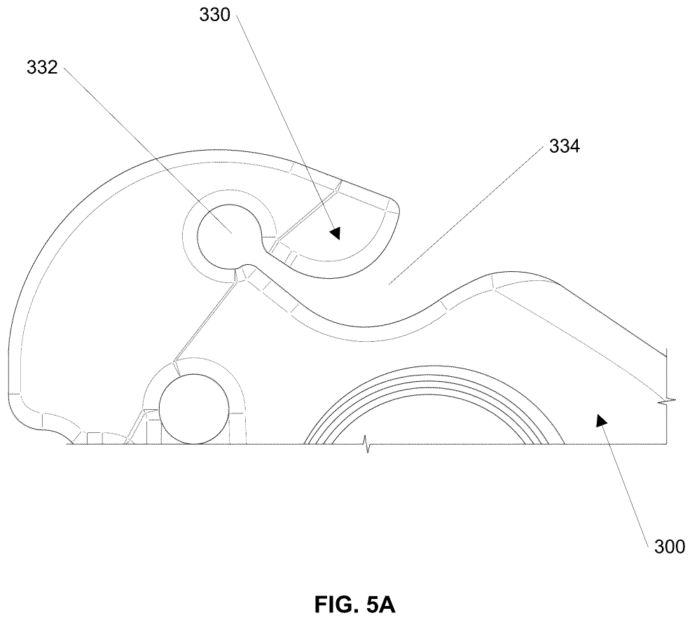

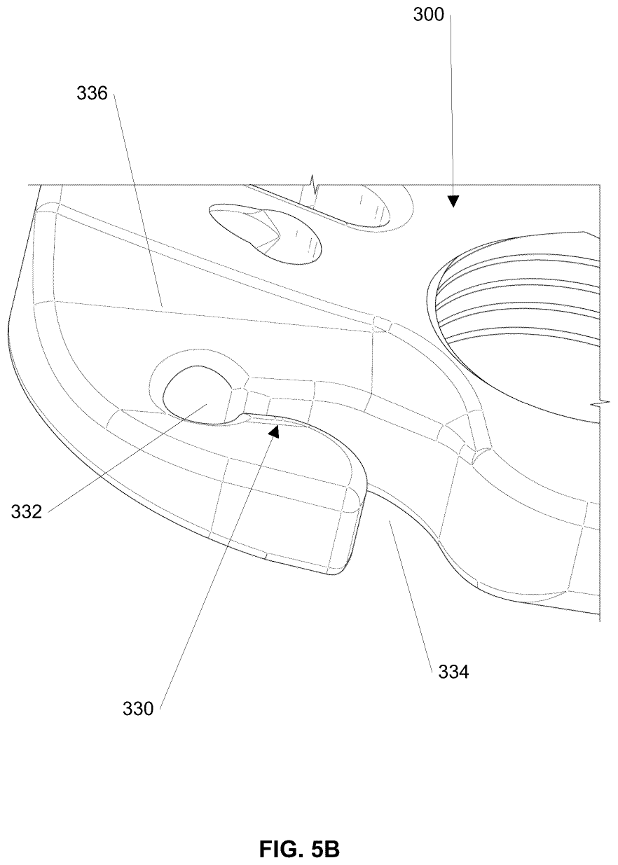

FIGS. 4A-4F and 5A-5B show close-up views of the suture attachments 320 and cleats 330 associated with bone plate 300, 300A. Specifically, FIGS. 4A-4F depict the suture attachments 320 and FIGS. 5A-5B depict the cleats 330. The suture attachments 320 and cleats 330 shown and detailed in FIGS. 4A-4F and 5A-5B may be any of the other suture attachments 120, 220 and cleats 130, 230 utilized in other embodiments of the bone plates 100, 100A, 200, 300, 300A.

The suture attachments 320 and cleats 330 may include a set of holes and tunnels on either end or both ends of the bone plate 300, 300A that accommodate a suture or other flexible device. The holes and tunnels may be configured in such a way that the suture or other flexible device can be routed through to create a situation where one end is free moving, and the other is rigid creating adjustability in one direction and locking in the other and opposite direction. This configuration may allow for soft tissue reattachment and deforming force neutralization.

The suture or other flexible device may be routed through the holes and tunnels to create a knot that is able to be pulled one way only. This configuration allows for the suture or other flexible device to be pulled freely toward the center of the plate while at the same time eliminates the ability to pull the other free end away from the plate, creating a locked or non-extendable suture or flexible device. FIG. 4A depicts an example of the suture holes 322A, 322B and slots 326, 328 that allow the suture to be attached to the bone plate 300, 300A and 4B depicts an example of the suture holes 322A, 322B or slots 326, 328 that allows a free end of the suture to pass through the bone plate 300, 300A. FIG. 4C depicts an example of the holes 322A, 322B and slots 326, 328 that allow for attachment of the free end of the suture or other flexible device. FIG. 4D depicts the suture slot 326 on the bottom or bone contacting side of the bone plate 300, 300A where the suture or flexible member is directed from one of the parallel suture holes 322A to the other suture hole 322B. FIG. 4E depicts a cross section of the parallel suture holes 322A, 322B that are drilled approximately perpendicular to the top surface of the bone plate 300, 300A which are sized approximately based on the size of the suture or other flexible member that is routed through. FIG. 4F depicts a cross-section view of the suture slot 326 that bisects one of the two suture holes 322A, 322B that are perpendicular to the top surface and serves as the path for the needle end of the suture or flexible member.

FIG. 4A-4F specifically depict a portion of the bone plate 300, 300A showing one of the suture attachments 320. FIGS. 4A and 4B show a dorsal surface 306 of the bone plate 300, 300A and FIGS. 4C and 4D show a volar surface 308 of the bone plate 300, 300A. The dorsal surface 306 may refer to a back side of the bone or limb, opposite the palm or anterior aspect. The volar surface 308 may refer to a palm-side or anterior aspect of the bone or limb, opposite the dorsal surface 306.

The suture attachment 320, shown in FIGS. 4A-4E, may include two suture holes 322A, 322B, a suture opening 324, and a suture slot 326. The suture holes 322A may have a parallel axis and the same diameter. The suture holes 322A, 322B may be oriented perpendicular to the dorsal surface 306 of the bone plate 300, 300A. The suture holes 322A may be spaced a distance apart and may extend through the thickness of the bone plate 300, 300A from the bone contacting surface to the opposite surface or the dorsal surface 306 to the volar surface 308. The suture holes may include a connecting suture hole 322A connected to the suture slot 326 and a second suture hole 322B. Additionally, the suture attachment 320 may include a connecting slot 328 between the connecting suture hole 322A and the second suture hole 322B. The connecting slot 328 may extend between the connecting suture hole 322A and the second suture hole 322B and be configured to direct a suture or flexible member through the bone plate.

The suture slot 326 may be co-axial to the suture holes 322A, 322B. The suture slot 326 may originate within the suture opening 324 and connects and bisects with one of the connecting suture hole 322A. The suture slot 326 may start on an outer surface of the edge of the bone plate 300, 300A, and terminate inside the connecting suture hole 322A. The suture slot 326 may create a tunnel on the bone contacting surface of the bone plate 300, 300A starting on the outside surface of the bone plate 300, 300A and terminating into the second suture hole 322B.

The suture or other flexible device may be routed through the suture opening 324 and the suture slot 326 from the outside of the bone plate 300, 300A and up away from the bone contacting surface through the connecting suture hole 322A. The suture may then be routed down through the adjacent parallel hole, the second suture hole 322B, and out through the connecting tunnel. The suture and flexible device may then be passed under the loop created by passing it between the first parallel suture holes 322A, 322B and along the top surface of the bone plate 300, 300A. While the bone plate 300, 300A depicts suture attachment 320 located on opposite ends and opposite sides of the bone plate 300, 300A, other numbers, configurations, and locations may be utilized for the suture attachment 320 configuration without departing from this invention.

FIGS. 5A and 5B specifically depict a portion of the bone plate 300, 300A showing one of the cleats 330. FIG. 5A depicts an example of a second set of holes and tunnels that allows for attachment of the free end of the suture or other flexible device. FIG. 5B depicts the second set of holes and tunnels and the undercut that allows for secure attachment of the free end of the suture or other flexible device. The cleat 330 may include a cleat hole 332 that is perpendicular to the volar surface 308 of the bone plate 300, 300A and a cleat slot 334 that connects the cleat hole 332 to the outer surface of the edge of the bone plate 300, 300A. FIG. 5B depicts a perspective side view of the cleat 330, showing the volar surface 308 of the bone plate 300, 300A. The cleat 330 may also include an undercut 336 for the cleat 330, as shown in FIG. 5B. The undercut 336 may be configured to allow the suture to be passed onto and through the cleat 330 while the bone plate 300, 300A is attached to the bone. While the bone plate 300, 300A depicts two cleats 330 located on opposite ends and opposite sides of the bone plate 300, 300A, other numbers, configurations, and locations may be utilized for the cleat 330 configuration without departing from this invention.

In other exemplary embodiments, and as described and present above, the present invention relates to a bone plate incorporating an integrated slip-knot feature that enables a suture or other flexible member to be routed through the bone plate 100, 100A, 200, 300, 300A in a manner that provides directional locking and self-fixation, creating a knotless soft-tissue fixation construct.

Conventional bone plates rely on external knot-tying or separate fixation components to secure sutures and soft tissues. In contrast, the bone plate 100, 100A, 200, 300, 300A and the slip-knot feature described herein integrates a series of holes, slots, and tunnels that are strategically oriented and geometrically configured to form a self-locking pathway for the suture. This pathway allows the suture or soft member to slide freely in one direction for tensioning, while locking against reverse movement once tension is applied, thereby forming a stable slip knot without the need for tying or external locking devices.

The slip-knot feature may include one or more entry and exit holes suture holes 322A, 322B, connected by angled or curved tunnels, openings, or slots 324, 326, 328 that guide the suture through the plate body. The geometry of these passages may be specifically designed to: 1) control frictional engagement between the suture and the internal surfaces of the plate; 2) promote directional locking by orienting the pathway so that tensile load in one direction increases the compressive interaction between the suture and the plate walls; and 3) prevent backsliding or loosening once the desired tension is achieved.

In one embodiment, the suture may be routed through a first entry suture hole 322A, across a tunnel or slot 326 angled relative to the plate surface, and then back through a second suture hole 322B or opening that positions the suture to cross itself within or on the surface of the plate. This self-crossing interaction may enhance locking behavior by creating a self-fixation mechanism, where the suture effectively binds against itself under tension. The design enables precise intraoperative control of tissue tension while eliminating the need for external knots, reducing operative time, and minimizing the potential for knot-related soft tissue irritation or bulk. The locking mechanism can be repeatedly adjusted before final fixation, offering the surgeon a means to fine-tune the construct tension for optimal biomechanical performance.

The novel aspects of the slip-knot feature and locking mechanism may include: 1) a system of integrated holes, slots, and tunnels oriented to create a self-locking or slip-knot pathway within a bone plate; 2) a mechanical design that allows unidirectional suture movement for tensioning and prevents reverse movement, eliminating the need for traditional knots; 3) a self-fixating construct, in which the suture locks on itself within or against the plate body; and 4) compatibility with various suture materials or soft members, enabling broad application across different fixation systems and anatomical sites. This integrated slip-knot feature transforms the bone plate into an active component of the tensioning mechanism, allowing surgeons to achieve secure, knotless soft-tissue fixation with enhanced precision and consistency.

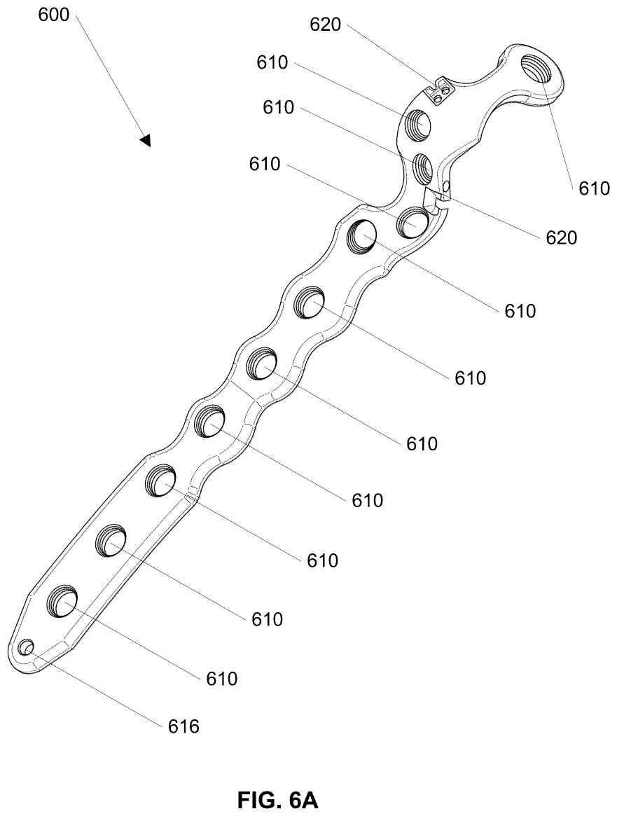

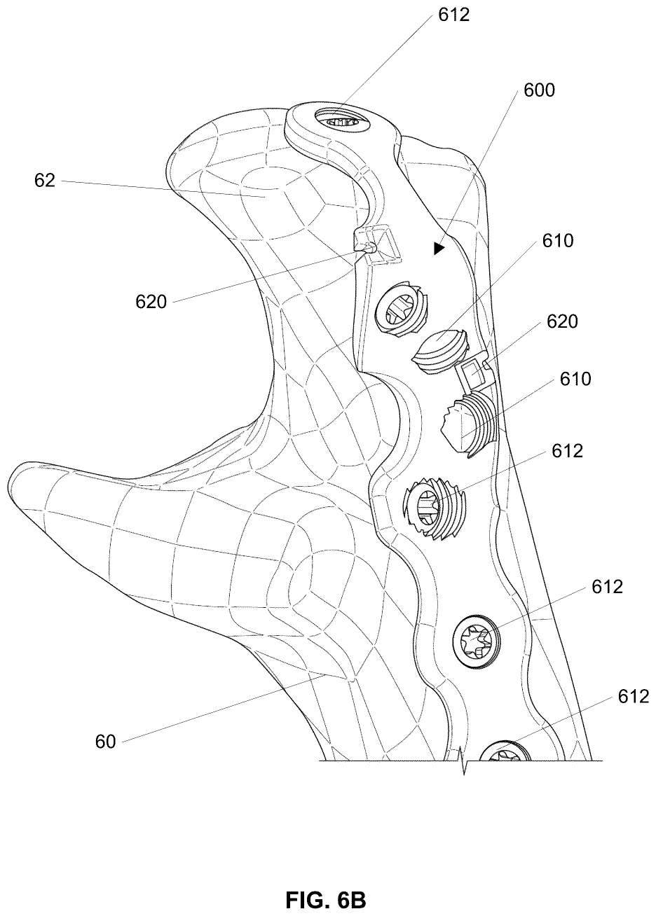

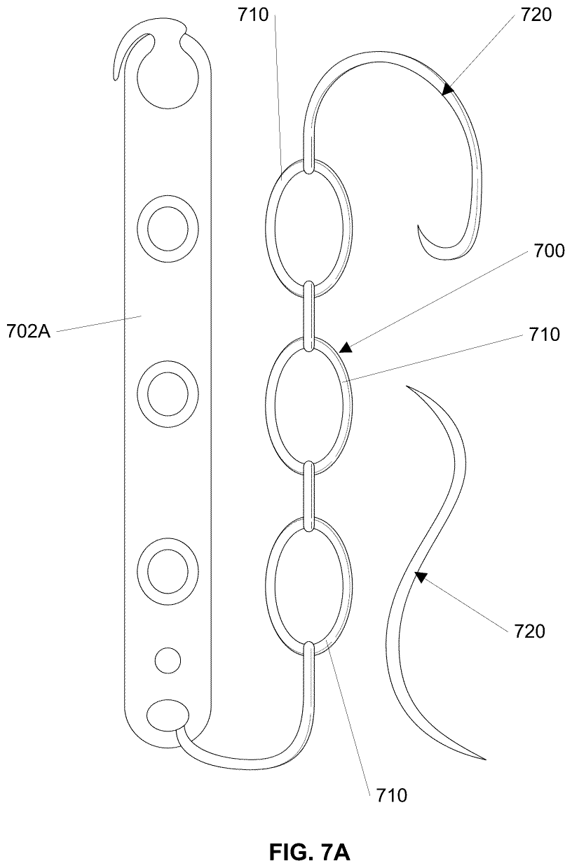

FIGS. 6A and 6B depict another embodiment of a bone plate 600 with multiple screw holes 610 and two suture attachment locations 620. Specifically, FIG. 6A depicts a perspective view of the bone plate 600, while FIG. 6B depicts a view of the bone plate 600 from FIG. 6B with the bone plate 600 attached via screws 612 on the lateral ulna bone or lateral olecranon. For the embodiment of FIGS. 6A and 6B, the features of the bone plate 600 are referred to using similar reference numerals under the “6XX” series of reference numerals, rather than “1XX” as used for the bone plate 100, 100A in the embodiments of FIGS. 1A-1C, “2XX” as used for the bone plate 200 in the embodiment of FIG. 2 and “3XX” as used for the bone plate 300, 300A in the embodiments of FIGS. 3A-3C. Accordingly, certain features of the bone plate 600 that were already described above with respect to the bone plate 100, 100A of FIGS. 1A-1C, the bone plate 200 of FIG. 2, and the bone plate 300, 300A of FIGS. 3A-3C may be described in lesser detail, or may not be described at all.

FIG. 6A specifically shows a bone plate 600 or a lateral ulna plate 600. The lateral ulna plate 600 may be intended to be placed on the lateral aspect of the proximal ulna and hook over the olecranon. The lateral ulna plate 600 may contain suture attachments 620 in at least two locations. The lateral ulna plate 600 may allow pre-attached sutures to be used to capture the triceps muscles and/or tendons to lock them to the lateral ulna plate 600 to facilitate healing. FIG. 6B specifically shows the lateral ulna plate 600 attached to the lateral aspect of the ulna bone 60 and wrapping around the olecranon 62. The lateral ulna plate 600 may also include various locking screws 612 extending through the various screw holes 610. FIG. 6B does not show any pre-attached suture.

As shown in FIGS. 6A and 6B, a lateral ulna plate 600 may be a bone fixation plate specifically configured for implantation on the lateral aspect of the ulna bone 60, particularly for the treatment of fractures involving the olecranon 62 and proximal ulna. The design of the lateral ulna plate 600 may provide rigid fixation while simultaneously minimizing soft-tissue irritation and plate prominence commonly associated with traditional posteriorly placed ulna plates.

Conventional olecranon plates are typically positioned along the posterior surface of the ulna, where minimal soft-tissue coverage often results in postoperative discomfort, hardware irritation, and the need for secondary removal. The lateral ulna plate 600 of the present invention may address these shortcomings by being anatomically contoured to the lateral surface of the proximal ulna 60, thereby reducing hardware prominence and improving patient comfort without compromising fixation strength.

The lateral ulna plate 600 may include a plurality of locking screw holes 610 arranged to accept standard or variable-angle locking screws 612 for rigid internal fixation of proximal ulna 60 and olecranon 62 fracture fragments. Additionally, the lateral ulna plate 600 may incorporate K-wire holes 616 for provisional fixation and intraoperative fragment reduction, enabling temporary stabilization during definitive screw placement.

A key feature of the lateral ulna plate 600 may include a slip-knot suture pathway or suture attachment 620, as previously described, within the plate body. This pathway or suture attachment 620 may consist of a series of strategically oriented holes, slots, and tunnels that allow a suture or flexible member to be routed through the plate in a slip-knot configuration. The feature may enable the surgeon to tension and secure the triceps or other soft-tissue attachments to the bone construct in a knotless, self-locking manner. By incorporating the slip-knot feature directly into the lateral ulna plate 600, the system allows precise control of the triceps extensor mechanism tension, reducing the risk of soft-tissue laxity or over-tensioning following fracture fixation.

The lateral positioning of the lateral ulna plate 600 may provide biomechanical advantages by placing the fixation construct in a zone that resists the predominant bending and torsional forces generated by the triceps during elbow extension. This orientation may enable rigid fixation of complex olecranon and proximal ulna fractures while maintaining a low-profile implant that is less palpable beneath the skin.

The novel aspects of the lateral ulna plate 600 may include: 1) a bone plate 600 anatomically contoured for placement on the lateral surface of the ulna, specifically for the treatment of olecranon and proximal ulna fractures; 2) reduced plate prominence and improved soft-tissue compatibility compared to posteriorly positioned plates; 3) integrated locking screw holes 610 to provide rigid internal fixation and K-wire holes 616 for provisional alignment and fixation; 4) incorporation of a slip-knot suture feature 620 within the plate, enabling knotless soft-tissue fixation and precise control of triceps tension; 5) enhanced biomechanical efficiency, allowing rigid fixation while minimizing soft-tissue irritation and potential for hardware removal. This novel lateral ulna plate 600 represents a significant advancement in the treatment of olecranon fractures by combining anatomically optimized fixation geometry with integrated soft-tissue tensioning capability, resulting in improved surgical workflow, biomechanical stability, and postoperative outcomes.

Although the bone plates described herein are illustrated in connection with various specific locations on the body, such as the lateral surface of the proximal ulna and olecranon region, the underlying design principles are not limited to any specific anatomical location. The bone plates, suture pathways, and slip-knot configurations of the present invention can be adapted for use in a variety of anatomical sites throughout the body where combined bone fixation and soft-tissue reattachment are desired. Such locations include, without limitation, the patella, tibial tuberosity, greater tuberosity of the humerus, malleolus, and other regions subjected to deforming or extensor mechanism forces. The plate geometry, size, curvature, and hole, tunnel, and slot configuration may be modified to conform to different bone contours while maintaining the same functional characteristics of knotless tensioning and controlled force neutralization.

A bone plating system may comprise a bone plate 100, 100A, 200, 300, 300A, 600 may be configured to conform to the anatomical region for which it is intended. The bone plate 100, 100A, 200, 300, 300A, 600 may be pre-contoured to match the surface geometry of bones such as the patella, olecranon, greater tuberosity of the humerus, or greater trochanter of the femur. In some embodiments, the bone plate 100, 100A, 200, 300, 300A, 600 may be symmetrical and suitable for either right or left application. In other embodiments, the bone plate 100, 100A, 200, 300, 300A, 600 may exhibit a side-specific, asymmetrical design to more precisely follow anatomical contours.

The bone plate 100, 100A, 200, 300, 300A, 600 may include a plurality of apertures designed to receive both locking and non-locking bone screws. These apertures may be distributed along the length or width of the plate in a pattern optimized for the specific fracture type and anatomical region, thereby providing the surgeon with flexibility in screw placement to address various fracture configurations.

The bone plate 100, 100A, 200, 300, 300A, 600 may be manufactured from a biocompatible material such as titanium or stainless steel. The bone plate 100, 100A, 200, 300, 300A, 600 may have a low-profile geometry to minimize soft tissue irritation postoperatively. The bone plate 100, 100A, 200, 300, 300A, 600 may include surface edges and transitions that are smoothed or radiused to further reduce interference with surrounding soft tissues, tendons, and ligaments.

One or more tunnels or channels may be machined directly into the body of the bone plate 100, 100A, 200, 300, 300A, 600. These channels may extend through the thickness of the bone plate 100, 100A, 200, 300, 300A, 600 and may be configured to receive a flexible member. The channels may be arranged to facilitate a slip-knot configuration, enabling the flexible member to pass in one direction and resist movement in the opposite direction when tension is applied. The orientation and position of the channels may vary depending on the anatomical location and the desired force vector to be redirected onto the bone plate 100, 100A, 200, 300, 300A, 600.

The bone plate 100, 100A, 200, 300, 300A, 600 may include a plurality of channels or tunnels machined through its thickness to guide and retain a flexible member. These channels may be configured to permit routing of the flexible member in a slip knot configuration, wherein tension applied to the free end of the flexible member causes the flexible member to cinch upon itself, removing slack and increasing compression across the associated soft tissue or fracture site.

The slip knot mechanism may be friction-based and may not require mechanical ratcheting or locking components. Instead, the interaction between the flexible member and the interior surface geometry of the channels may enable resistance to back-sliding once tension is applied. The flexible member may be routed through a sequence of channels that create a circuitous path, encouraging directional friction and mechanical locking as the member tightens on itself.

Multiple sets of such channels or tunnels may be included on a single bone plate 100, 100A, 200, 300, 300A, 600. The positioning of the channels and tunnels may vary depending on the intended anatomic application, such as to accommodate soft tissue redirection points around the patella, olecranon, greater tuberosity, or greater trochanter. This modularity may enable the bone plating system to be adapted to specific fracture patterns or biomechanical demands. The spatial configuration of the channels may also be designed to align the flexible member in a desired force vector, redirecting the pull of the extensor mechanism onto the bone plate 100, 100A, 200, 300, 300A, 600 itself.

The bone plating system may further include a flexible member configured to be routed through the integrated channels or tunnels within the bone plate 100, 100A, 200, 300, 300A, 600. The flexible member may be fabricated from a variety of biocompatible materials, including but not limited to high-strength suture, braided polymer, wire, or composite materials. The flexible member may be provided in multiple cross-sectional geometries, including round, flat, or combinations thereof, depending on the clinical application and desired flexibility, tensile strength, and handling characteristics.

A unique feature of the flexible member may be the incorporation of a plurality of loops or openings that are integral to its cross-sectional body. These loops may be spaced at regular or variable intervals along the length of the member and are designed to engage with a cleat or anchor point on the bone plate. The flexible member configuration may allow the surgeon to select a specific loop or opening during implantation, thereby controlling the amount of tension exerted across the soft tissue structure or fracture site. The ability to incrementally tension the construct facilitates fine-tuned force modulation and accommodates patient-specific anatomical or fracture variables.

The cleat or anchor may be an integral structural feature of the bone plate and may be configured to accept and retain a selected loop or opening of the flexible member. In one embodiment, the cleat may comprise a hook-and-ramp design, in which a first end is narrower and sloped or tapered to allow guided insertion of the loop, while the second end includes a geometry configured to capture the loop in a frictional or snap-fit engagement. This snap-fit or mechanical retention mechanism may create a secure, knotless construct that resists disengagement under physiological loading. The design may ensure that once the flexible member is tensioned and secured, forces from the extensor mechanism are effectively redirected onto the bone plate and away from the fracture site.

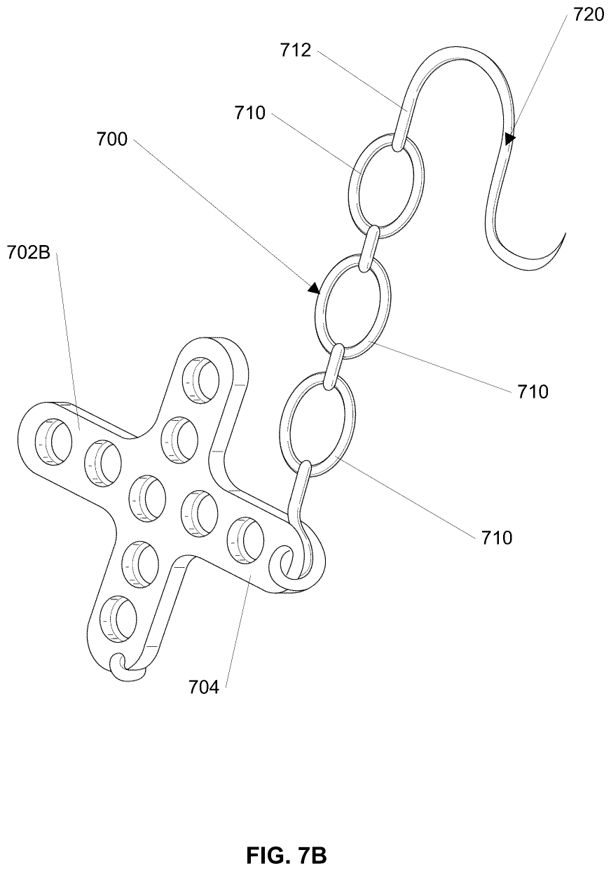

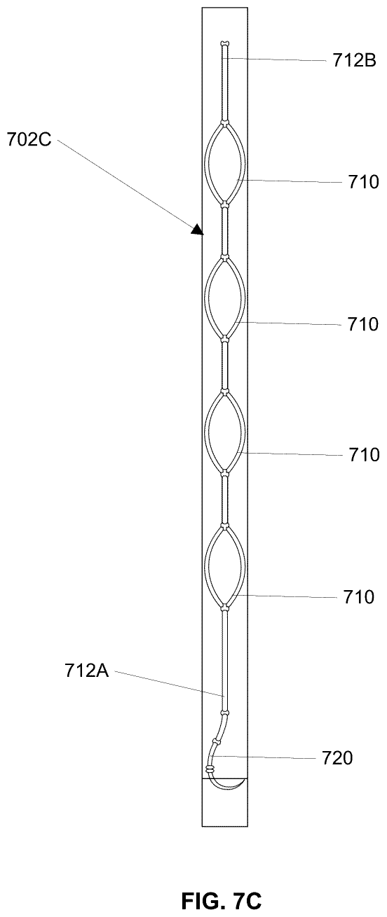

FIGS. 7A-7C depict various exemplary embodiments related to a loop suture 700 designed to improve precision, consistency, and control in soft-tissue fixation procedures involving tendons, ligaments, and muscles. The loop suture 700 may include one or more integrated, pre-formed loops 710 along a length, with each loop 710 functioning as a selectable tensioning point that enables a surgeon to incrementally adjust the amount of tension applied to soft tissue during implantation.

The loop suture 700 of the present invention may allow the user to “dial in” tension by engaging specific loops 710, thereby achieving reproducible and quantifiable tissue compression. The system minimizes variability, reduces the risk of overtightening, and eliminates bulky knots that can irritate surrounding tissue.

The loop suture 700 may be provided with a pre-attached surgical needle 720 to facilitate passage through soft tissue and may attach at its opposite end to a bone plate or implant via a fixed connection or an adjustable slip-knot configuration. In certain embodiments, the loops 710 may be evenly spaced and integrally formed from the same continuous strand of high-strength suture material, ensuring uniform tensile characteristics and simplifying intraoperative handling.

This innovation enables precise, repeatable, and adjustable soft-tissue tensioning, improving fixation reliability and surgical efficiency compared to traditional suture techniques. The invention relates to a loop suture 700 designed to enhance control and precision in soft tissue fixation procedures involving tendons, ligaments, and muscles. The loop suture 700 may include one or more integrated suture loops 710 formed along a length, with each loop 710 configured to enable the surgeon to selectively adjust the amount of tension applied to the soft tissue during implantation.

Unlike conventional sutures, which require manual knot tying or tensioning after passage through the tissue, the loop suture 700 of the present invention allows the surgeon to “dial in” a precise amount of tension by engaging one or more of the pre-formed loops 710. Each loop 710 functions as a mechanical intermediary that converts incremental suture displacement into a controlled increase or decrease in tissue compression or tension. This configuration provides reproducible, quantifiable tension control and minimizes the risk of overtightening or tissue strangulation.

The loop suture 700 may include a pre-attached surgical needle 720 at one end to facilitate soft tissue passage. The opposing end of the loop suture 700 may be configured for attachment to a bone plate or implant, either in a fixed connection (e.g., crimped, tied, or captured within a locking feature) or in a slip-knot configuration, allowing the loop suture 700 to dynamically adjust tension during or after fixation.

In some embodiments, the plurality of loops 710 may be evenly spaced and integrally formed from the same continuous suture material, providing a consistent tensile profile along the length of the construct. The configuration allows for multiple levels of soft tissue engagement and tension control using a single suture strand, thereby simplifying the procedure and improving intraoperative efficiency.

The novel aspects of the loop suture 700 may include: 1) the incorporation of one or more built-in loops along the length of the suture; 2) the ability to incrementally control or “dial in” soft tissue tension by selecting or engaging specific loops; 3) compatibility with fixed or adjustable attachment configurations to bone plates or other fixation devices; and 4) optional pre-attached needle to streamline surgical workflow.

The loop suture 700 may enable surgeons to achieve precise, repeatable, and adjustable tensioning of soft tissues, improving fixation reliability and reducing the technical variability associated with traditional suture and knot-tying techniques.

FIG. 7A depicts a bone plate 702A with loop suture 700 for use with the bone plating system. The loop suture 700 may be attached in a fixed manner or can be attached in a slip knot configuration (as described above). The loop suture 700 may have a needle 720 attached to it. The loops 710 on the loop suture 700 may be used to attach a free end 712 of the loop suture 700 to the plates cleat. The loop suture 700 may allow for the user to determine the amount of tension or slack to put on the loop suture 700.

FIG. 7B depicts a patella plate 702B with loop suture 700 for use with the bone plating system. The loop suture 700 may be attached to one arm 704 of a multiple arm plate or patella plate 702B. The loop suture 700 in this embodiment may have a needle 720 attached to the free end 712. Multiple loop sutures 700 may be attached to the patella plate 702B. The loops 710 on the free end 712 of the loop suture 700 may mate with cleats on the patella plate 702B which could accommodate multiple fixation points.

FIG. 7C depicts another loop suture 700C for use with the bone plating system. The loop suture 700C may include a needle 720 attached to one free end 712A and a straight length of suture attached to the other free end 712B. The loop suture 700C may include multiple loops 710. The multiple loops may be located at multiple points in between the free ends 712A, 712B and in various intervals. The free end 712B without the needle 720 may be attached to a bone plate in a locked or slip knot configuration. The loops 710 may allow the surgeon to create fixation of tissue in more or less tension.

Although the loop sutures described herein are illustrated with numbers of loops, the underlying design principles are not limited to any specific number of loops and configurations.

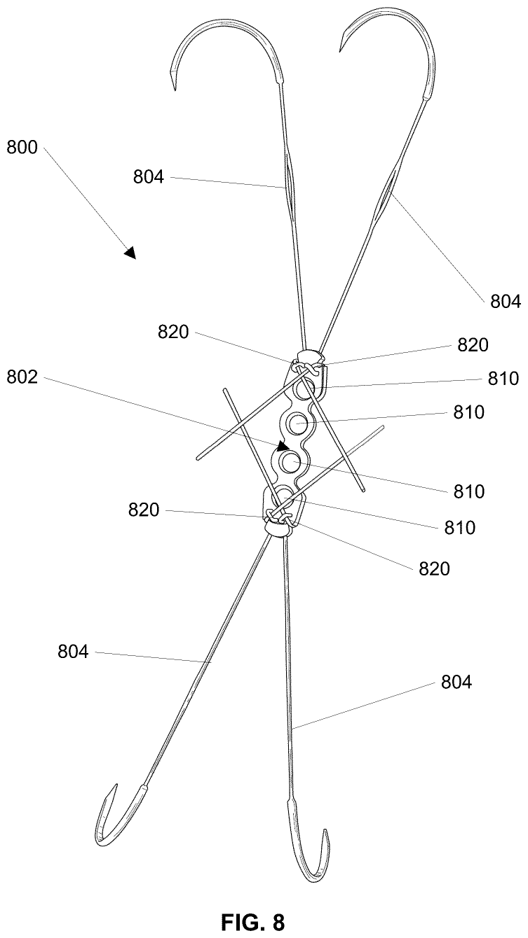

FIG. 8 depicts a bone plate system 800 that includes a bone plate 802 with sutures 804 that are pre-attached to the bone plate 802 in four different places. The bone plate 802 may be a bendable plate for anatomic fit. The sutures 804 may be configured on the bone plate 802 at the attachment points 820 in a slip-knot configuration. The sutures 804 may be pulled freely in the direction toward the center of the bone plate 802, but may be locked when force is exerted in a direction away from the bone plate 802. The bone plate 802 shown in FIG. 8 includes four holes 810 for screw attachment, however, the bone plate 802 may include various other numbers of holes 810, such as one, two, three, or four or more holes 810 for screw attachment.

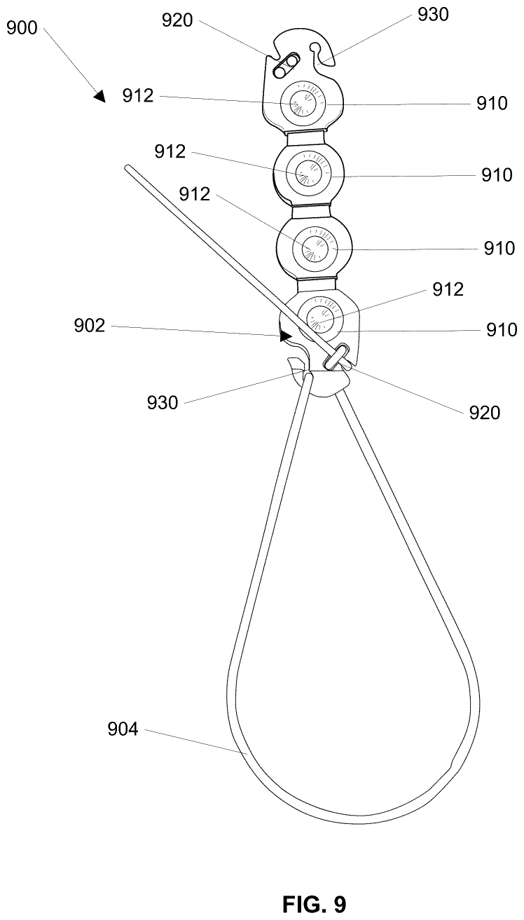

FIG. 9 depicts another bone plate system 900 that includes a bone plate 902 with a suture 904 attached to the bone plate 902 in a one-way pull suture slip-knot configuration. The suture 904 may attached to the bone plate 902 via an attachment point 920 and a cleat 930. The bone plate 902 may include four holes 910 and four screws 912. The screws 912 may be attached to the bone plate 902 via matching conical locking threads. The screws 912 may include screw head thread that match the bone plate threads. The bone plate 902 shown in FIG. 9 includes four holes 910 for screw 912 attachment, however, the bone plate 902 may include various other numbers of holes 910, such as one, two, three, or four or more holes 910 for screw 912 attachment.

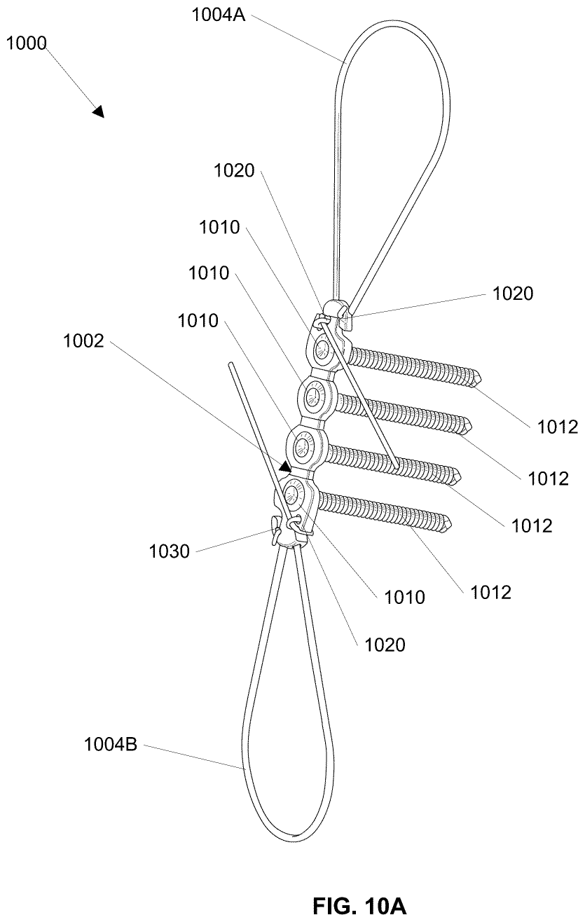

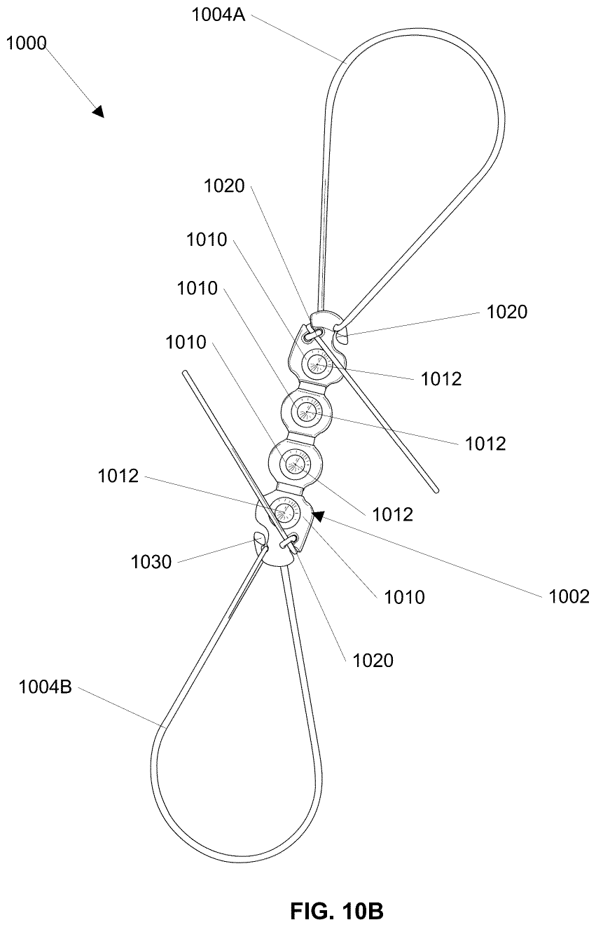

FIGS. 10A and 10B depict another bone plate system 1000 that includes a bone plate 1002 with two sutures 1004A, 1004B attached to the bone plate 1002 in a one-way pull suture slip-knot configuration. The sutures 1004A, 1004B may attached to the bone plate 1002 via an attachment point 1020 and a cleat 1030. The bone plate 1002 may include four holes 1010 and four screws 1012. The screws 1012 may be attached to the bone plate 1002 via matching conical locking threads. The screws 1012 may include screw head thread that match the bone plate threads. The bone plate 1002 shown in FIGS. 10A and 10B includes four holes 1010 for screw 1012 attachment, however, the bone plate 1002 may include various other numbers of holes 1010, such as one, two, three, or four or more holes 1010 for screw 1012 attachment.

The present disclosure is disclosed above and in the accompanying drawings with reference to a variety of examples. The purpose served by the disclosure, however, is to provide examples of the various features and concepts related to the disclosure, not to limit the scope of the invention. It is to be understood that the invention is not limited in its application to the details of construction and the arrangement of the components set forth herein. The invention is capable of other embodiments and of being practiced or being carried out in various ways. Variations and modifications of the foregoing are within the scope of the present invention. It should be understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention.

While the preferred embodiments of the invention have been shown and described, one skilled in the relevant art will recognize that numerous variations and modifications may be made to the examples described above without departing from the scope of the present disclosure. Thus, the spirit and scope of the invention should be construed broadly as set forth in the appended claims.

The present technology is disclosed above and in the accompanying drawings with reference to a variety of embodiments. The purpose served by the disclosure, however, is to provide an example of the various features and concepts related to the technology, not to limit its scope. One skilled in the relevant art will recognize that numerous variations and modifications may be made to the embodiments described above without departing from the scope of the present invention, as defined by the appended claims.

For the avoidance of doubt, the present application includes at least the subject matter described in the following numbered Clauses:
Clause 1. A bone plate for orthopedic fixation, comprising: a plate body having a dorsal surface, a volar surface, and a bone-contacting surface; a plurality of screw holes extending through the plate body and configured to receive bone screws for fixation to a bone; a first set of suture holes and tunnels formed through the plate body and configured to allow a suture or other flexible member to pass therethrough and attach to the bone plate, the first set being arranged such that one end of the suture passes through soft tissue and an opposite end of the suture is movable to remove slack; wherein the first set of holes and tunnels is oriented and dimensioned to permit movement of the suture in a first direction for tensioning and to restrict movement in an opposite direction, thereby maintaining unidirectional tension between the soft tissue and the bone plate; and a second set of holes and tunnels configured to receive and secure a free end of the suture or flexible member to the bone plate.
Clause 2. The bone plate according to Clause 1, wherein the first set of suture holes comprises a first suture hole and a second suture hole extending generally perpendicular to the dorsal surface and substantially parallel to one another.
Clause 3. The bone plate according to Clause 2, further comprising a connecting tunnel extending between the first suture hole and the second suture hole and configured to direct the suture or flexible member through the plate body.
Clause 4. The bone plate according to Clause 2, wherein the first suture hole and the second suture hole extend through the thickness of the plate body from the bone-contacting surface to the opposite surface.
Clause 5. The bone plate according to Clause 1, further comprising a suture slot positioned on an edge of the plate and extending from an exterior surface of the plate body to intersect one of the suture holes, the slot providing a pathway for entry of the suture into the plate.
Clause 6. The bone plate according to Clause 5, wherein the suture slot is substantially co-axial with the suture holes and forms part of the unidirectional slip-knot pathway.
Clause 7. The bone plate according to Clause 1, wherein the first set of suture holes, tunnels, and slots form a self-locking slip-knot pathway that allows the suture to slide freely in one direction during tensioning while resisting reverse motion once tension is applied.
Clause 8. The bone plate according to Clause 7, wherein the slip-knot pathway is configured such that the suture crosses itself within or along the plate body to increase frictional engagement and self-fixation.
Clause 9. The bone plate according to Clause 1, wherein the second set of holes and tunnels forms a cleat configured to anchor the free end of the suture.
Clause 10. The bone plate according to Clause 9, wherein the cleat includes a cleat hole oriented perpendicular to the volar surface and a cleat slot extending from the cleat hole to an outer edge of the plate.
Clause 11. The bone plate according to Clause 10, wherein the cleat further includes an undercut recessed beneath the volar surface to retain the suture after fixation.
Clause 12. The bone plate according to Clause 1, wherein the plate body comprises one or more threaded screw holes configured to receive locking screws and one or more smooth holes configured to receive non-locking screws.
Clause 13. The bone plate according to Clause 1, wherein the plate body is low-profile and anatomically contoured to conform to a bone surface.
Clause 14. The bone plate according to Clause 1, wherein the plate body is bendable at one or more junctions between screw holes to accommodate irregular bone geometries.
Clause 15. The bone plate according to Clause 1, wherein the plate includes at least one suture attachment positioned adjacent a plate end and at least one cleat positioned on an opposing plate end.
Clause 16. The bone plate according to Clause 1, wherein the plate body comprises a material selected from the group consisting of titanium, stainless steel, cobalt-chromium alloy, and biocompatible polymer composites.
Clause 17. A method of securing soft tissue to a bone, comprising: providing the bone plate according to Clause 1; attaching the bone plate to a bone using one or more bone screws; routing a suture or flexible member through the first set of holes and tunnels in the plate so that a first end of the suture passes through the soft tissue; tensioning the suture in a first direction to remove slack while preventing reverse motion through the slip-knot pathway; and securing a free end of the suture to the bone plate through the second set of holes and tunnels.
Clause 18. The method according to Clause 17, wherein the suture is routed such that it crosses itself within or along the plate to create a self-locking configuration.
Clause 19. The method according to Clause 17, wherein the bone plate is positioned on a dorsal, volar, or lateral surface of the bone depending on the fracture location.
Clause 20. The method according to Clause 17, wherein the bone plate is applied to an anatomical site selected from the group consisting of the olecranon, proximal ulna, patella, tibial tuberosity, malleolus, scapula, and greater tuberosity of the humerus.
Clause 21. A lateral ulna bone fixation plate, comprising: a plate body anatomically contoured to conform to a lateral surface of a proximal ulna and configured to wrap partially around an olecranon; a plurality of screw holes extending through the plate body and configured to receive locking or non-locking screws for fixation of olecranon and proximal-ulna fracture fragments; at least one suture attachment feature comprising one or more holes, tunnels, or slots configured to receive a suture or flexible member and form a slip-knot pathway that allows the suture to move in a first direction for tensioning and resist movement in an opposite direction, thereby maintaining a knotless self-locking configuration for soft-tissue fixation; and wherein the plate body is positioned on the lateral aspect of the ulna to reduce soft-tissue irritation and provide rigid fixation while minimizing implant prominence relative to posteriorly positioned plates.
Clause 22. The lateral ulna bone fixation plate according to Clause 21, wherein the plate body includes a proximal portion configured to hook over the olecranon to provide proximal fixation and tendon capture.
Clause 23. The lateral ulna bone fixation plate according to Clause 21, wherein the suture attachment feature is configured to enable a pre-attached suture to engage and secure a triceps muscle or tendon to the plate to facilitate healing.
Clause 24. The lateral ulna bone fixation plate according to Clause 21, wherein the slip-knot pathway comprises a series of strategically oriented holes, tunnels, and slots configured to guide the suture through the plate and promote unidirectional movement for tensioning.
Clause 25. The lateral ulna bone fixation plate according to Clause 24, wherein the geometry of the holes, tunnels, and slots is oriented to increase frictional engagement between the suture and the plate walls, thereby enhancing self-locking behavior.
Clause 26. The lateral ulna bone fixation plate according to Clause 24, wherein the suture crosses itself within or along the plate body to produce a self-binding interaction that resists loosening once tension is applied.
Clause 27. The lateral ulna bone fixation plate according to Clause 21, wherein the plate body includes two or more suture attachment locations disposed along opposite ends of the plate.
Clause 28. The lateral ulna bone fixation plate according to Clause 21, wherein the plate body further includes one or more K-wire holes configured for provisional fixation and intraoperative reduction of fracture fragments.
Clause 29. The lateral ulna bone fixation plate according to Clause 21, wherein the plurality of screw holes comprise variable-angle locking holes configured to receive screws at a range of insertion angles.
Clause 30. The lateral ulna bone fixation plate according to Clause 21, wherein the plate body is formed of a biocompatible metal or composite material selected from the group consisting of titanium, stainless steel, cobalt-chromium alloy, and reinforced polymer composites.
Clause 31. The lateral ulna bone fixation plate according to Clause 21, wherein the plate body is low-profile and includes radiused or smoothed edges to minimize interference with surrounding soft tissue.
Clause 32. The lateral ulna bone fixation plate according to Clause 21, wherein the plate body is configured to provide biomechanical resistance to bending and torsional forces generated by the triceps during elbow extension.
Clause 33. The lateral ulna bone fixation plate according to Clause 21, wherein the slip-knot pathway enables adjustable intraoperative tensioning of the suture prior to final fixation.
Clause 34. The lateral ulna bone fixation plate according to Clause 21, wherein the plate geometry, curvature, and hole pattern are modularly configurable to conform to different bone surfaces while maintaining knotless tensioning capability.
Clause 35. The lateral ulna bone fixation plate according to Clause 21, wherein the plate body further includes identifiable dorsal and volar surfaces, the volar surface comprising a bone-contacting surface adapted to interface with the ulna.
Clause 36. The lateral ulna bone fixation plate according to Clause 21, wherein the plate is adaptable for use at other anatomical sites subject to deforming or extensor forces, including the patella, tibial tuberosity, greater tuberosity of the humerus, malleolus, and scapula.
Clause 37. The lateral ulna bone fixation plate according to Clause 21, wherein the slip-knot suture pathway and plate configuration together enable combined fracture fixation and soft-tissue reattachment within a single integrated implant.
Clause 38. A method of treating an olecranon or proximal-ulna fracture, comprising: positioning the lateral ulna bone fixation plate according to Clause 21 on a lateral surface of the proximal ulna such that the plate wraps partially around the olecranon; fixing the plate to the bone using a plurality of bone screws; routing a suture through the slip-knot pathway of the plate and through a triceps tendon or muscle; tensioning the suture in a first direction to secure the soft tissue against the plate while preventing reverse movement; and locking the suture in place without forming an external knot.
Clause 39. The method according to Clause 38, wherein the plate is applied to a lateral olecranon region to reduce posterior hardware prominence and minimize postoperative soft-tissue irritation.
Clause 40. The method according to Clause 38, wherein the bone plate and slip-knot pathway are adapted for use in other anatomical regions requiring combined bone fixation and soft-tissue reattachment.
Clause 41. A bone fixation plate, comprising: a plate body anatomically contoured to conform to a surface of a bone and configured to provide rigid fixation of one or more bone fragments; a plurality of screw holes extending through the plate body and configured to receive locking or non-locking bone screws for securing the plate to the bone; at least one suture pathway formed within the plate body and comprising one or more holes, tunnels, or slots configured to receive a suture or flexible member, the suture pathway defining a slip-knot configuration that permits the suture to move in a first direction for tensioning and restricts movement in an opposite direction, thereby providing unidirectional, knotless locking of the suture relative to the plate body; and wherein the plate body is configured to enable simultaneous bone fixation and soft-tissue reattachment, allowing controlled tensioning of a tendon, ligament, or muscle relative to the bone without requiring external knot-tying.
Clause 42. The bone fixation plate according to Clause 41, wherein the plate body includes a low-profile, anatomically contoured shape adapted to conform to a selected region of a skeletal structure.
Clause 43. The bone fixation plate according to Clause 41, wherein the suture pathway comprises a series of holes, tunnels, and slots arranged to form a self-locking, unidirectional slip-knot pathway.
Clause 44. The bone fixation plate according to Clause 43, wherein the geometry of the holes, tunnels, and slots promotes frictional engagement between the suture and the plate body to resist reverse movement once tension is applied.
Clause 45. The bone fixation plate according to Clause 41, wherein the suture pathway is configured to route the suture such that it crosses itself within or along the plate body, producing a self-binding interaction that enhances locking strength.
Clause 46. The bone fixation plate according to Clause 41, further comprising at least one cleat or anchor feature configured to capture a free end of the suture in a knotless, self-locking manner.
Clause 47. The bone fixation plate according to Clause 41, wherein the plate body further includes one or more K-wire holes to facilitate provisional fixation and intraoperative fracture reduction.
Clause 48. The bone fixation plate according to Clause 41, wherein the plate body is formed of a biocompatible metallic or composite material selected from the group consisting of titanium, stainless steel, cobalt-chromium alloy, and fiber-reinforced polymer composites.
Clause 49. The bone fixation plate according to Clause 41, wherein the plate is adaptable for multiple anatomical locations including the ulna, olecranon, patella, tibial tuberosity, greater tuberosity of the humerus, malleolus, or scapula.
Clause 50. A bone plating system, comprising: a bone plate having a plurality of screw holes configured to receive screws for fixation to a bone; at least one integrated suture pathway within the bone plate, the pathway including one or more holes, tunnels, or slots configured to guide a flexible member through the plate; at least one cleat or anchor feature on the bone plate configured to receive and retain a selected portion of the flexible member; and the flexible member comprising a strand having a plurality of integral loops or openings spaced along its length, each loop configured to engage with the cleat or anchor feature to establish a selectable amount of tension between the flexible member and the bone plate, wherein engagement of one of the loops with the cleat forms a knotless, self-locking fixation construct that maintains tension under physiological loading.
Clause 51. The bone plating system according to Clause 51, wherein the flexible member is fabricated from a biocompatible material selected from the group consisting of high-strength suture, braided polymer, metallic wire, or composite fiber.
Clause 52. The bone plating system according to Clause 51, wherein the flexible member has a cross-sectional geometry selected from round, flat, or ribbon-like shapes.
Clause 53. The bone plating system according to Clause 51, wherein the plurality of loops are integrally formed within the flexible member and spaced at regular or variable intervals along its length.
Clause 54. The bone plating system according to Clause 51, wherein the cleat or anchor feature comprises a hook-and-ramp design having a tapered insertion end and a retaining end configured for snap-fit or frictional engagement with one of the loops.
Clause 55. The bone plating system according to Clause 54, wherein the cleat or anchor feature is integral with the bone plate body and positioned to allow guided insertion of the selected loop under tension.
Clause 56. The bone plating system according to Clause 51, wherein the flexible member includes a pre-attached surgical needle at a first end configured for passage through soft tissue, and a second end configured to engage with the bone plate in either a fixed or slip-knot configuration.
Clause 57. The bone plating system according to Clause 51, wherein the flexible member is configured such that tension can be incrementally adjusted by selecting different loops for engagement with the cleat.
Clause 58. The bone plating system according to Clause 51, wherein the bone plate includes a slip-knot suture pathway formed by a series of angled holes or tunnels that allow the flexible member to move freely in one direction for tensioning while resisting movement in an opposite direction.
Clause 59. The bone plating system according to Clause 58, wherein the slip-knot pathway is oriented to promote directional locking by increasing compressive interaction between the flexible member and the internal plate surfaces when loaded.
Clause 60. The bone plating system according to Clause 51, wherein the bone plate is anatomically contoured and configured for placement on a bone surface selected from the group consisting of the lateral ulna, olecranon, patella, tibial tuberosity, greater tuberosity of the humerus, and malleolus.
Clause 61. The bone plating system according to Clause 51, wherein the bone plate includes one or more K-wire holes for provisional fixation and intraoperative reduction of fracture fragments.
Clause 62. The bone plating system according to Clause 51, wherein the flexible member is pre-attached to the bone plate at one or more attachment locations.
Clause 63. The bone plating system according to Clause 51, wherein the flexible member is configured to provide fine-tuned force modulation by permitting loop-by-loop tension adjustment.
Clause 64. The bone plating system according to Clause 51, wherein the cleat or anchor feature is configured such that the flexible member, once engaged, redirects tensile forces from an attached soft tissue onto the bone plate rather than the fracture site.
Clause 65. The bone plating system according to Clause 51, wherein the combination of the plate, cleat, and looped flexible member forms a knotless, adjustable soft-tissue reattachment system integrated within the bone fixation construct.
Clause 66. The bone plating system according to Clause 51, wherein the bone plate is low-profile and bendable, allowing contouring to the patient's anatomy while maintaining locking screw fixation.
Clause 67. The bone plating system according to Clause 51, wherein the flexible member and bone plate together provide a single-construct fixation system capable of simultaneously securing bone fragments and reattaching soft tissue under controlled, adjustable tension.
Clause 68. A method of securing soft tissue to bone, comprising: attaching a bone plate according to Clause 51 to a bone using screws; routing a flexible member through a soft-tissue structure and through the plate's suture pathway; selecting a loop along the flexible member and engaging it with the plate's cleat or anchor feature; applying tension to the flexible member to achieve a desired compression level; and locking the loop to the cleat to form a knotless, self-locking fixation construct that maintains the applied tension during healing.
Clause 69. A loop suture for orthopedic fixation, comprising: a continuous suture strand formed of a biocompatible flexible material; a plurality of pre-formed loops integrally formed along the length of the suture strand, each loop configured to engage a fixation element of a bone plate or anchor; and wherein selection of one or more of the pre-formed loops enables incremental adjustment of tension applied to a soft-tissue structure during implantation, thereby providing controlled, repeatable, and knotless soft-tissue fixation.
Clause 70. The loop suture according to Clause 69, wherein the suture includes a pre-attached surgical needle at one end to facilitate tissue passage.
Clause 71. The loop suture according to Clause 69, wherein the pre-formed loops are evenly spaced and integrally formed from the same continuous strand.
Clause 72. The loop suture according to Clause 69, wherein the loops are dimensioned to engage a cleat, hook, or anchor feature of a bone plate in a frictional or snap-fit engagement.
Clause 73. The loop suture according to Clause 69, wherein the material comprises UHMWPE, polyester, or nylon.
Clause 74. A bone plate for orthopedic fixation, comprising: a plate body having a plurality of screw holes for bone fixation; a system of holes, tunnels, or slots extending through the plate body, the system being oriented to define a self-locking suture pathway; and wherein the suture pathway is configured to permit a suture or flexible member to move in a first direction for tensioning and to resist movement in an opposite direction, thereby forming a knotless slip-knot fixation construct.
Clause 75. The bone plate according to Clause 74, wherein the pathway includes a first entry hole, a second exit hole, and an angled tunnel connecting the holes such that a suture routed therethrough crosses itself within or against the plate body to enhance frictional locking.
Clause 76. The bone plate according to Clause 74, wherein the geometry of the pathway increases compressive interaction between the suture and the plate when tensile load is applied.
Clause 77. The bone plate according to Clause 74, further comprising a cleat or anchor feature adjacent the pathway to receive a loop suture and maintain tension in a knotless configuration.
Clause 78. The bone plate according to Clause 74, wherein the plate is constructed of titanium, stainless steel, or a biocompatible polymer composite.
Clause 79. A bone fixation plate configured for placement on a lateral surface of the proximal ulna, comprising: a plate body anatomically contoured to the lateral aspect of the ulna; a plurality of locking screw holes arranged for rigid fixation of olecranon and proximal-ulna fracture fragments; at least one K-wire hole for provisional fixation; and an integrated suture pathway configured as a slip-knot feature that permits unidirectional movement of a suture for tensioning and resists reverse movement, enabling knotless reattachment of the triceps tendon to the bone.
Clause 80. The bone fixation plate according to Clause 79, wherein the suture pathway is oriented to oppose the tensile vector of the triceps mechanism.
Clause 81. The bone fixation plate according to Clause 79, wherein the plate includes smooth and radiused edges to reduce soft-tissue irritation.
Clause 82. The bone fixation plate according to Clause 79, wherein the plate integrates the loop suture according to Clause 1 and the slip-knot pathway according to Clause 6 to provide adjustable, knotless fixation.
Clause 83. The bone fixation plate according to Clause 79, wherein the lateral positioning of the plate provides reduced prominence relative to posterior fixation plates.

What is claimed is:

1. A lateral ulna bone fixation plate, comprising:
a plate body anatomically contoured to conform to a lateral surface of a proximal ulna and configured to wrap partially around an olecranon;
a plurality of screw holes extending through the plate body and configured to receive locking or non-locking screws for fixation of olecranon and proximal-ulna fracture fragments;
at least one suture attachment feature comprising one or more holes, tunnels, or slots configured to receive a suture or flexible member and form a slip-knot pathway that allows the suture to move in a first direction for tensioning and resist movement in an opposite direction, thereby maintaining a knotless self-locking configuration for soft-tissue fixation; and
wherein the plate body is positioned on the lateral aspect of the ulna to reduce soft-tissue irritation and provide rigid fixation while minimizing implant prominence relative to posteriorly positioned plates.

2. The lateral ulna bone fixation plate according to claim 1, wherein the plate body includes a proximal portion configured to hook over the olecranon to provide proximal fixation and tendon capture.

3. The lateral ulna bone fixation plate according to claim 1, wherein the suture attachment feature is configured to enable a pre-attached suture to engage and secure a triceps muscle or tendon to the plate to facilitate healing.

4. The lateral ulna bone fixation plate according to claim 1, wherein the slip-knot pathway comprises a series of strategically oriented holes, tunnels, and slots configured to guide the suture through the plate and promote unidirectional movement for tensioning.

5. The lateral ulna bone fixation plate according to claim 4, wherein a geometry of the holes, tunnels, and slots is oriented to increase frictional engagement between the suture and one or more plate walls, thereby enhancing self-locking behavior.

6. The lateral ulna bone fixation plate according to claim 4, wherein the suture crosses itself within or along the plate body to produce a self-binding interaction that resists loosening once tension is applied.

7. The lateral ulna bone fixation plate according to claim 1, wherein the plate body includes two or more suture attachment locations disposed along opposite ends of the plate.

8. The lateral ulna bone fixation plate according to claim 1, wherein the plate body further includes one or more K-wire holes configured for provisional fixation and intraoperative reduction of fracture fragments.

9. The lateral ulna bone fixation plate according to claim 1, wherein the plurality of screw holes comprise variable-angle locking holes configured to receive screws at a range of insertion angles.

10. The lateral ulna bone fixation plate according to claim 1, wherein the plate body is formed of a biocompatible metal or composite material selected from the group consisting of titanium, stainless steel, cobalt-chromium alloy, and reinforced polymer composites.

11. The lateral ulna bone fixation plate according to claim 1, wherein the plate body is low-profile and includes radiused or smoothed edges to minimize interference with surrounding soft tissue.

12. The lateral ulna bone fixation plate according to claim 1, wherein the plate body is configured to provide biomechanical resistance to bending and torsional forces generated by a triceps during elbow extension.

13. The lateral ulna bone fixation plate according to claim 1, wherein the slip-knot pathway enables adjustable intraoperative tensioning of the suture prior to final fixation.

14. The lateral ulna bone fixation plate according to claim 1, wherein a plate geometry, curvature, and hole pattern are modularly configurable to conform to different bone surfaces while maintaining knotless tensioning capability.

15. The lateral ulna bone fixation plate according to claim 1, wherein the plate body further includes identifiable dorsal and volar surfaces, the volar surface comprising a bone-contacting surface adapted to interface with the ulna.

16. The lateral ulna bone fixation plate according to claim 1, wherein the slip-knot suture pathway and plate configuration together enable combined fracture fixation and soft-tissue reattachment within a single integrated implant.

17. A method of treating an olecranon or proximal-ulna fracture, comprising:
positioning the lateral ulna bone fixation plate according to claim 1 on a lateral surface of the proximal ulna such that the plate wraps partially around the olecranon;
fixing the plate to the bone using a plurality of bone screws; routing a suture through the slip-knot pathway of the plate and through a triceps tendon or muscle;
tensioning the suture in a first direction to secure soft tissue against the plate while preventing reverse movement; and
locking the suture in place without forming an external knot.

18. The method according to claim 17, wherein the plate is applied to a lateral olecranon region to reduce posterior hardware prominence and minimize postoperative soft-tissue irritation.

19. A bone fixation plate configured for placement on a lateral surface of a proximal ulna, comprising:
a plate body anatomically contoured to the lateral surface of the proximal ulna;
a plurality of locking screw holes arranged for rigid fixation of olecranon and proximal-ulna fracture fragments;
at least one K-wire hole for provisional fixation; and
an integrated suture pathway configured as a slip-knot feature that permits unidirectional movement of a suture for tensioning and resists reverse movement, enabling knotless reattachment of a triceps tendon to the bone.

20. The bone fixation plate according to claim 19, wherein the suture pathway is oriented to oppose a tensile vector of a triceps mechanism.

21. The bone fixation plate according to claim 19, wherein the plate includes smooth and radiused edges to reduce soft-tissue irritation.

22. The bone fixation plate according to claim 19, wherein the lateral positioning of the plate provides reduced prominence relative to posterior fixation plates.
